(19) United States
(12) Patent Application Publication
(10) Pub. No.: US 2024/0342156 A1
(43) Pub. Date: Oct. 17, 2024

(54) METHODS FOR TREATING PAIN

(71) Applicant: Xenon Pharmaceuticals Inc., Burnaby (CA)

(72) Inventors: James Philip Johnson, JR., Vancouver (CA); Gregory N. Beatch, Vancouver (CA)

(73) Assignee: Xenon Pharmaceuticals Inc., Burnaby (CA)

(21) Appl. No.: 18/415,139

(22) Filed: Jan. 17, 2024

Related U.S. Application Data
(63) Continuation of application No. 17/121,305, filed on Dec. 14, 2020, now abandoned.
(60) Provisional application No. 62/948,010, filed on Dec. 13, 2019.

(51) Int. Cl. A61K 31/472; A61K 9/00; A61P 23/00

(52) U.S. Cl. CPC A61K 31/472; A61K 9/0053; A61P 23/00

(57) ABSTRACT

In certain embodiments, the present disclosure is directed to methods for treating pain in a subject, such as a human, wherein the methods comprise orally administering a therapeutically effective amount of N-[4-(6-fluoro-3,4-dihydro-1H-isoquinolin-2-yl)-2,6-dimethylphenyl]-3,3-dimethylbutanamide (Compound A), to the subject in need thereof. The present disclosure is further directed to various improved methods of therapy and administration of Compound A.

### 1. BACKGROUND

Pain is a major medical problem that affects nearly 120 million people in the United States. Drug therapy is the mainstay of management for acute and chronic pain in all age groups, including neonates, infants, and children. Pain drugs are classified by the American Pain Society into three main categories: 1) non-opioid analgesics (e.g., acetaminophen) and non-steroidal anti-inflammatory drugs (NSAIDs) (e.g., aspirin), 2) opioid analgesics, and 3) co-analgesics.

Sodium channel blockers have been shown to be useful in the treatment of pain, including acute, chronic, inflammatory, and neuropathic pain (see, e.g., Wood, J. N., et al., J. Neurobiol. (2004), 61(1), 55-71). Preclinical evidence demonstrates that sodium channel blockers can suppress neuronal firing in peripheral and central sensory neurons, and it is via this mechanism that they are considered to be useful for relieving pain.

Many pain sufferers, particularly those suffering from chronic pain, cannot be treated effectively. The consequences of ineffective pain treatment include reduced mobility, limited function, poor sleep, and an overall low quality of life. There remains a need in the art for novel and effective treatments of pain, including neuropathic pain and nociceptive pain, such as inflammatory pain. The present disclosure addresses this need by providing compositions and methods and uses for treating pain, and offers other related advantages.

Citation of any reference in the Background section of this application is not to be construed as an admission that such reference is prior art to the present application.

### 2. SUMMARY

The present disclosure describes certain methods and uses for the small molecule N-[4-(6-Fluoro-3,4-dihydro-TH-isoquinolin-2-yl)-2,6-dimethylphenyl]-3,3-dimethylbutanamide (herein referred to as “Compound A”).

In one embodiment, the present disclosure is directed to a method of treating pain in a subject (preferably, a mammal, such as a human) in need thereof, comprising administering a therapeutically effective amount of Compound A to the subject. In certain instances, the pain treated by the administration of Compound A is nociceptive pain, neuropathic pain, or a combination thereof. In certain embodiments, the pain treated by the administration of Compound A is nociceptive pain, such as radicular pain, somatic pain, visceral pain, soft tissue pain, inflammatory pain, post-operative pain, or a combination thereof, particularly post-operative pain.

In an additional embodiment, the method of treating pain comprising administering a therapeutically effective amount of Compound A further comprises enhancing the opening of a Kv7 potassium channel in the subject (e.g., human).

In another embodiment, the present disclosure is directed to a method of opening or enhancing the opening of a Kv7 potassium channel in a subject (preferably, a mammal, such as a human), comprising administering an effective amount of Compound A to the subject, wherein the subject is suffering from pain such as the various types of pain described herein, including nociceptive pain, neuropathic pain, or a combination thereof, particularly inflammatory pain.

In some aspects, the Kv7 potassium channel is one or more of Kv7.2, Kv7.3, Kv7.4, or Kv7.5. In certain instances, the opening or enhanced opening of one or more of the Kv7.2, Kv7.3, Kv7.4, or Kv7.5 potassium channels is selective over Kv7.1. In other instances, the method comprises opening or enhanced opening of the Kv7.2/Kv7.3 (KCNQ2/3) potassium channel.

In one embodiment, the present disclosure provides a method of treating pain in a subject (preferably, a mammal, such as a human) in need thereof, wherein Compound A is administered (preferably orally) to the subject. In certain instances, the administration to the subject comprises a dose of 2 to 200 mg of Compound A per administration. In other instances, the administration to the subject comprises a dose of 5-1000 mg per day. In further instances, the administration to the subject comprises a dose of 0.05-20 mg/kg, such as 0.1-10 mg/kg.

In some embodiments of the present methods and uses, Compound A is orally administered to the subject (preferably, a mammal, such as a human) from between about 30 minutes before to about 2 hours after eating a meal, for example, Compound A may be orally administered to the subject during a meal or within 15 minutes after eating a meal.

In certain embodiments, the present disclosure provides a method of treating pain in a subject (preferably, a mammal, such as a human) in need thereof, comprising administering (e.g., orally) a therapeutically effective amount of Compound A to the subject in combination with one or more additional analgesic agents, such as an opioid analgesic.

In additional embodiments, the present disclosure provides a method of reducing the dose (e.g., a maintenance dose) of an opioid analgesic administered to a subject (preferably, a mammal, such as a human) in need thereof comprising administering (e.g., orally) a therapeutically effective amount of Compound A to the subject, for example, whereby the effective amount of Compound A reduces the dose of the opioid analgesic needed to achieve pain relief in the subject.

Compound A is a small molecule currently being developed for the treatment of seizure disorders, and its use as a potassium channel modulator is disclosed in U.S. Pat. Nos. 8,293,911 and 8,993,593 as well as U.S. application Ser. Nos. 16/409,684 and 16/410,851, the disclosures of which are hereby incorporated by reference in their entireties.

These and other aspects of this disclosure will be apparent upon reference to the following detailed description. To this end, various references are set forth herein which describe in more detail certain background information and procedures and are each hereby incorporated by reference in their entirety.

### 3. BRIEF DESCRIPTION OF THE DRAWINGS

FIG. 1 shows results of the acetic acid induced mouse model of visceral pain showing nociceptive events (y-axis) and vehicle, 1 mg/kg, 3 mg/kg, and 10 mg/kg dosing of Compound A (x-axis) for Study 1 (top left), Study 2 (top right), the combination of Study 1 and Study 2 (bottom left), and PK/PD correlation (bottom right) showing a PK/PD correlation between Compound A concentrations in brain and plasma to the observed efficacy.

FIG. 2 shows results from the electronic von Frey test on non-lesion and lesion paws in groups of rats on Day 13, prior to treatment, showing force inducing paw-withdrawal (g) (y-axis) and the future treatment to be administered: vehicle, 8 mg/kg, 16 mg/kg, and 24 mg/kg Compound A, 20 mg/kg retigabine, and 128 mg/kg morphine p.o. dosing (x-axis). Paired student's t-test (versus non-lesioned paw): NS=Not Significant; *=p<0.05; **=p<0.01; ***=p<0.001.

FIG. 3 shows results from the electronic von Frey test on lesioned paw (tactile allodynia evaluation on Day 14 and Day 18) in rats showing variation (delta from baseline) of force inducing paw-withdrawal (g) (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 4 shows results from the cold plate test on lesioned paw (thermal allodynia evaluation on Day 14 at 2 h) in rats showing latency to the first paw-withdrawal (s) (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 5 shows results from the cold plate test on lesioned paw (thermal allodynia evaluation on Day 14 at 2 h) in rats showing number of withdrawal responses (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 6 shows results from the cold plate test on lesioned paw (thermal allodynia evaluation on Day 14 at 2 h) in rats showing total duration of withdrawal responses (s) (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 7 shows results from the cold plate test on lesioned paw (thermal allodynia evaluation on Day 18 at 2 h) in rats showing latency to the first paw-withdrawal (s) (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 8 shows results from the cold plate test on lesioned paw (thermal allodynia evaluation on Day 18 at 2 h) in rats showing number of withdrawal responses (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

FIG. 9 shows results from the cold plate test on lesioned paw (thermal allodynia evaluation on Day 18 at 2 h) in rats showing total duration of withdrawal responses (s) (y-axis) and vehicle, 8 mg/kg, 16 mg/kg, and 24 mg/kg Compound A, 20 mg/kg retigabine, and 128 mg/kg morphine p.o. dosing (x-axis). Inter-group comparison (versus vehicle (p.o.)): NS=Not Significant; *=p<0.05; **=p<0.01; ***=p<0.001.

### 4. DETAILED DESCRIPTION

The present disclosure relates to novel and improved methods and uses for Compound A, particularly for treatment of pain by administering Compound A to a subject (preferably, a mammal, such as a human) in need thereof by oral administration or by other routes.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 4.1. Definitions

As used in the specification and appended claims, unless specified to the contrary, the following terms and abbreviations have the meaning indicated:

“Compound A” refers to the compound having the following formula:

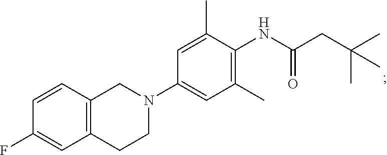 [SMILES: Cc1cc(N2CCc3cc(F)ccc3C2)cc(C)c1NC(=O)CC(C)(C)C]

and having a chemical name of N-[4-(6-fluoro-3,4-dihydro-TH-isoquinolin-2-yl)-2,6-dimethylphenyl]-3,3-dimethylbutanamide. Preparation of Compound A and its use as a Kv7.2/Kv7.3 (KCNQ2/3) opener is disclosed in U.S. Pat. Nos. 8,293,911 and 8,993,593 as well as U.S. application Ser. Nos. 16/409,684 and 16/410,851. Compound A potentiates and enhances opening of the voltage-gated potassium channels Kv7.2 and Kv7.3 (Kv7.2/Kv7.3), which are important in controlling neuronal excitability. Compound A is used in the methods and uses described herein.

“Acute pain” as used herein means pain that has a recent onset. Acute pain commonly declines over a short time (e.g., days, hours, or minutes) and follows injury to the body, and generally disappears when the bodily injury heals.

“Breakthrough pain” as used herein means a transitory increase in pain above the baseline or background pain experienced by a patient. In this context, “baseline pain” means the pain that is experienced or reported by a patient as the average pain intensity experienced for 12 or more hours.

“Chronic pain” as used herein means pain persisting for at least a week. Typically, chronic pain persists for three to six months or longer.

The phrase “in combination” as used herein in the context of administering Compound A refers to the simultaneous or sequential administration of Compound A with one or more additional therapeutic agents, such as one or more other pain treatments, regimens, or analgesic agents. For example, administering Compound A in combination with another therapeutic agent means that Compound A may be administered with another therapeutic agent simultaneously or sequentially in separate unit dosage forms (e.g., as part of a multiple dosage regimen) or together in a single unit dosage form. If the additional therapeutic agent and Compound A are administered sequentially, then this could be within a period of time up to 24 hours from the other, such 0.25, 0.5, 1, 2, 3, 4, 5, 6, 7, 8, 9, 10, 11, 12, 13, 14, 15, 16, 17, 18, 18, 20, 21, 22, 23, or 24 hours or less from the other.

“Pain” as used herein refers to all categories of pain and includes, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g., musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

“Therapeutically effective amount” as used herein refers to an amount of Compound A that is sufficient to treat the stated disease, disorder, or condition or have the desired stated effect on the disease, disorder, or condition or one or more mechanisms underlying the disease, disorder, or condition in a subject. In certain embodiments, when Compound A is administered for the treatment of pain, therapeutically effective amount refers an amount of Compound A which, upon administration to a subject, treats or ameliorates pain in the subject, or exhibits a detectable therapeutic effect in the subject that results in reduction in pain. Changes in pain experienced by a patient can be measured through the use of a pain rating scale, and such scales are used in daily clinical practice to measure pain intensity. Commonly used pain measurement scales include the Visual Analog Scale (VAS), the Graphic Rating Scale (GRS), the Simple Descriptor Scale (SDS), the Numerical Rating Scale (NRS), and the Faces Rating Scale (FRS). All of these scales have been documented as being valid measures of pain intensity.

“Treatment” as used herein refers to therapeutic applications associated with administering Compound A that ameliorate the indicated disease, disorder, or condition (e.g., pain) or one or more underlying mechanisms of said disease, disorder, or condition, including slowing or stopping progression of the disease, disorder, or condition or one or more of the underlying mechanisms in a subject. In certain embodiments, when Compound A is administered for the treatment of pain, treatment refers to therapeutic applications to slow or stop the increase of pain (i.e., to stabilize the level of pain) and/or reduction or elimination of pain. In some embodiments, the treatment of pain comprising the administration of Compound A is accompanied by an alteration of the cellular activity of one or more Kv7 potassium channels (e.g., Kv7.2, Kv7.3, Kv7.4, and/or Kv7.5, particularly Kv7.2 and/or Kv7.3, optionally over Kv7.1) toward a normal level that would be observed in the absence of the pain.

“Under fed conditions” refers to the condition of having consumed food during the time period between from about 4 hours prior to the oral administration of an effective amount (e.g., within the therapeutically effective dose range) of Compound A to about 4 hours after the administration of Compound A. The food may be a solid, liquid, or mixture of solid and liquid food with sufficient bulk and fat content that it is not rapidly dissolved and absorbed in the stomach. In some instances, the food is a meal, such as breakfast, lunch, dinner or, alternatively, baby food (e.g., formula or breast milk). The therapeutically effective amount of Compound A may be orally administered to the subject, for example, between about 30 minutes before to about 2 hours after eating a meal, most advantageously, Compound A is orally administered during a meal or within 15 minutes after eating a meal.

“Under fasted conditions” refers to the condition of not having consumed food during the time period between from at least 4 hours prior to the oral administration of a therapeutically effective amount of Compound A to about 4 hours after administration of Compound A.

### 4.2. Embodiments

In some embodiments, the present disclosure is directed to a method of treating pain in a subject (preferably, a mammal, such as a human) in need thereof, comprising administering (e.g., orally) a therapeutically effective amount of Compound A to the subject. In certain instances, the pain treated by administering Compound A is nociceptive pain, neuropathic pain, or a combination thereof.

In some instances, the pain is nociceptive pain, such as radicular pain, somatic pain, visceral pain, soft tissue pain, inflammatory pain, or a combination thereof, particularly inflammatory pain, including inflammatory pain associated with an inflammatory disease or condition, such as organ transplant rejection; reoxygenation injury resulting from organ transplantation (see Grupp et al., J. Mol, Cell Cardiol. 31:297-303 (1999)) including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory bowel diseases, such as ileitis, ulcerative colitis, Barrett's syndrome, and Crohn's disease; inflammatory lung diseases, such as asthma and adult respiratory distress syndrome; inflammatory diseases of the eye, including corneal dystrophy, trachoma, onchocerciasis, uveitis, sympathetic ophthalmitis, and endophthalmitis; chronic inflammatory disease of the gum, including gingivitis and periodontitis; tuberculosis; leprosy; inflammatory diseases of the kidney, including uremic complications, glomerulonephritis and nephrosis; inflammatory disease of the skin, including sclerodermatitis, psoriasis and eczema; inflammatory diseases of the central nervous system, including chronic demyelinating diseases of the nervous system, multiple sclerosis, AIDS-related neurodegeneration, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, glaucoma, retinopathy, nephropathy (such as microaluminuria and progressive diabetic nephropathy), gangrene of the feet, atherosclerotic coronary arterial disease, peripheral arterial disease, foot ulcers, joint problems, and a skin or mucous membrane complication (such as an infection, a shin spot, a candidal infection or necrobiosis lipoidica diabeticorum), immune-complex vasculitis, and systemic lupus erythematosus (SLE); inflammatory disease of the heart, such as cardiomyopathy, ischemic heart disease hypercholesterolemia, and pericarditis; as well as various other diseases that can have significant inflammatory components, including preeclampsia, chronic liver failure, brain and spinal cord trauma, and cancer. The present methods and uses of Compound A can also be used to treat pain associated with an inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines.

In certain embodiments, the pain is neuropathic pain, including neuropathic pain selected from pain associated with spinal cord injury, spinal or brain stroke, multiple sclerosis, cancer, shingles, post-herpetic neuralgia, erythromelalgia (including inherited erythromelalgia), chemotherapy-induced neuropathy, oxaliplatin-induced neuropathy, trigeminal neuralgia, phantom pain, phantom limb pain, radiculopathy, complex regional pain syndrome, causalgia, reflex sympathetic dystrophy, lower back pain, peripheral nerve trauma, herpes virus infection, diabetes mellitus, diabetic neuropathy, plexus avulsion, neuroma, limb amputation, vasculitis, chronic alcoholism, human immunodeficiency virus (HIV) infection, uremia, vitamin deficiency, pelvic pain, or a combination thereof. In some embodiments, the neuropathic pain is chronic neuropathic pain, such as pain resulting from injury to the peripheral or central nervous tissue. In some embodiments, the neuropathic pain is a neuropathy, such as one of those described herein.

In some embodiments, the neuropathic pain is selected from pain associated with spinal cord injury, spinal or brain stroke, post-herpetic neuralgia, erythromelalgia (including inherited erythromelalgia), trigeminal neuralgia, radiculopathy, complex regional pain syndrome, causalgia, reflex sympathetic dystrophy, peripheral nerve trauma, diabetic neuropathy, plexus avulsion, neuroma, vasculitis, or a combination thereof.

In certain embodiments, the neuropathic pain is selected from pain associated with shingles, multiple sclerosis, cancer, chemotherapy-induced neuropathy, oxaliplatin-induced neuropathy, herpes virus infection, diabetes mellitus, human immunodeficiency virus (HIV) infection, hypothyroidism, uremia, or a combination thereof, particularly pain associated with multiple sclerosis or cancer. In certain embodiments, the neuropathic pain is selected from pain associated with shingles or herpes virus infection. In some embodiments, the neuropathic pain is selected from pain associated with cancer, chemotherapy-induced neuropathy, or oxaliplatin-induced neuropathy.

In some embodiments, the neuropathic pain is selected from pain associated with phantom pain, phantom limb pain, lower back pain, limb amputation, chronic alcoholism, vitamin deficiency, pelvic pain, or a combination thereof, particularly phantom pain, phantom limb pain, or pain associated with limb amputation.

In certain instances, the pain treated by administering a therapeutically effective amount of Compound A to the subject (preferably, a mammal, such as a human) is acute pain. In some embodiments, the pain is chronic pain. Such administration may be, e.g., by oral, sublingual, buccal, occur, otic, vaginal, rectal, cutaneous, topical, or transdermal administration; by intravenous, intramuscular, intrathecal, or subcutaneous injection; or by implantation.

In some instances, the pain treated by administering a therapeutically effective amount of Compound A to the subject (preferably, a mammal, such as a human) is mild, moderate, or severe pain. In certain embodiments, the pain is moderate or severe pain, or moderate to severe pain. Such administration may be, e.g., by oral, sublingual, buccal, occur, otic, vaginal, rectal, cutaneous, topical, or transdermal administration; by intravenous, intramuscular, intrathecal, or subcutaneous injection; or by implantation.

In certain instances, the pain treated by administering (e.g., orally) a therapeutically effective amount of Compound A to the subject (e.g., a human) is associated with a disease state or other condition, such as cancer pain, rheumatic pain, arthritic pain, bone pain, labor pain, myocardial infarction pain, pancreatic pain, colic pain, post-operative pain, headache pain, muscle pain, pain associated with a periodontal disease (including gingivitis and periodontitis), or a combination thereof. In some embodiments, the pain is of tumor origin. In other embodiments, the pain is of non-tumor origin. In certain embodiments, the pain is associated with a migraine, including migraine without aura (“common migraine”), migraine with aura (“classic migraine”), migraine without headache, basilar migraine, familial hemiplegic migraine, migrainous infarction, migraine with prolonged aura, or a combination thereof.

In some embodiments, the pain treated by administering (e.g., orally) a therapeutically effective amount of Compound A to the subject (e.g., a human) is breakthrough pain.

In some embodiments, the method of treating pain by administering a therapeutically effective amount of Compound A comprises enhancing the opening of a Kv7 potassium channel in the subject (preferably, a mammal, such as a human).

In certain embodiments, the present disclosure provides a method or use comprising opening or enhancing the opening of a Kv7 potassium channel, such as the Kv7.2, Kv7.3, Kv7.4, and/or Kv7.5 potassium channel, particularly the Kv7.2/Kv7.3 (KCNQ2/3) potassium channel in a subject in need thereof by administering an effective amount of Compound A. In some of such embodiments, the subject suffers from pain, such as the types of pain described herein, including neuropathic pain or nociceptive pain, such as inflammatory pain.

In certain instances, the method or use described herein comprises selectively opening or enhancing the opening of a Kv7 potassium channel, such as one or more of Kv7.2, Kv7.3, Kv7.4, or Kv7.5 over Kv7.1. In some embodiments, the method or use is selective for Kv7.2, over Kv7.1. In other embodiments, the method or use is selective for Kv7.3, over Kv7.1. In yet other embodiments, the method or use is selective for Kv7.4, over Kv7.1. In yet further other embodiments, the method or use is selective for Kv7.5, over Kv7.1. In certain embodiments, the method or use is selective for Kv7.2 and Kv7.3, over Kv7.1. In certain embodiments, the method or use is selective for Kv7.2 and Kv7.3 over other Kv7 potassium channels. In certain embodiments, the method or use is selective for Kv7.2 and Kv7.3 over Kv7.4 and Kv7.5.

As an alternative to oral administration, in certain instances other routes of administration of Compound A can be employed in the methods and used described herein, such as parenteral administration. Parenteral administration routes include subcutaneous, intravenous, intramuscular, intra-articular, intra-synovial, intrasternal, intrathecal, intrahepatic, intralesional, and intracranial injection or infusion techniques or by implantation. For example, Compound A can be administered by injection, such as by intravenous, intramuscular, intrathecal, or subcutaneous injection. In certain embodiments, the above-discussed doses of Compound A are intended for oral administration and can be converted to doses suitable for parenteral administration, including administration by injection, by reducing the oral dose, for example by about half.

Other administration routes suitable for administration of Compound A according to the methods and uses described herein include sublingual and buccal (e.g., with a film or other composition that dissolves in the mouth under the tongue or on the inside of the cheek), ocular (e.g., eye drops), otic (e.g., by ear drops), oral or nasal inhalation (e.g., by insufflation or nebulization), cutaneous or topical (e.g., by creams or lotions), or transdermal (e.g., by skin patches). Besides oral administration, other enteral administration routes can be used for Compound A, including vaginal and rectal (e.g., by ointment, suppository, enema).

The presently described methods and uses involving administering Compound A for treating pain may also include administering Compound A in combination with one or more additional therapeutic agents, such as one or more other pain treatments, regimens, or analgesic agents. For instance, in some embodiments, the present disclosure provides a method of treating pain in a subject (e.g., a human) in need thereof, comprising administering (e.g., orally) a therapeutically effective amount of Compound A to the subject in combination with one or more additional analgesic agents.

In some embodiments, the one or more additional analgesic agents include opioid analgesics, such as opioid agonists, mixed agonist-antagonists, or partial agonists including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, and tramadol, including mixtures of any of the foregoing and pharmaceutically acceptable salts of any of the foregoing. In particular embodiments, the additional analgesic agent includes an opioid agonist, such as buprenorphine, codeine, hydrocodone, hydromorphone, methadone, morphine, oxycodone, oxymorphone, tilidine, and tramadol including mixtures of any of the foregoing and pharmaceutically acceptable salts of any of the foregoing. In some embodiments, the additional analgesic agent is oxycodone or a pharmaceutically acceptable salt thereof, such as oxycodone HCl.

On other embodiments the one or more additional analgesic agents include non-opioid treatments, such as aspirin; acetaminophen; non-steroidal anti-inflammatory drugs (“NSAIDS”), e.g., ibuprofen, ketoprofen, naproxen, etc.; N-methyl-D-aspartate (NMDA) receptor antagonists, e.g., a morphinan such as dextromethorphan or dextrorphan, or ketamine; cyclooxygenase-2 inhibitors (“COX-II inhibitors”), such as celecoxib, rofecoxib, and etoricoxib; and/or glycine receptor antagonists.

In some embodiments, administering Compound A in combination with one or more additional analgesic agents permits a reduction in the dosage of the additional analgesic agent without a reduction in the level of pain relief or analgesic efficacy provided. For instance, in some embodiments, the present disclosure provides a method of treating pain in a subject (e.g., a human) in need thereof, comprising administering (e.g., orally) a therapeutically effective amount of Compound A to the subject in combination with an amount of one or more additional analgesic agents, wherein the amount of the additional analgesic agent is less than the amount of the additional analgesic agent that would be needed to achieve the same or a similar level of pain relief or analgesic efficacy in the absence of administering Compound A. In certain of such embodiments, the one or more additional analgesic agents is an opioid analgesic, such as buprenorphine, codeine, hydrocodone, hydromorphone, methadone, morphine, oxycodone, oxymorphone, tilidine, and tramadol including mixtures of any of the foregoing and pharmaceutically acceptable salts of any of the foregoing. In some embodiments, the additional analgesic agent is oxycodone or a pharmaceutically acceptable salt thereof, such as oxycodone HCl.

In related embodiments, the present disclosure provides a method of reducing the dose (e.g., maintenance dose) of an opioid analgesic administered to a subject (preferably, a mammal, such as a human) in need thereof comprising administering (e.g., orally) a therapeutically effective amount of Compound A to the subject, for example, whereby the effective amount of Compound A offsets the reduction in the dose of the opioid analgesic such that the level of pain relief or analgesic efficacy experienced by the subject is maintained. In certain of such embodiments, the opioid analgesic is selected from buprenorphine, codeine, hydrocodone, hydromorphone, methadone, morphine, oxycodone, oxymorphone, tilidine, and tramadol, including mixtures of any of the foregoing and pharmaceutically acceptable salts of any of the foregoing. In some embodiments, the opioid analgesic is oxycodone or a pharmaceutically acceptable salt thereof, such as oxycodone HCl.

In additional embodiments, the present disclosure provides a method of reducing the dose (e.g., a maintenance dose) of an opioid analgesic administered to a subject (preferably, a mammal, such as a human) in need thereof comprising administering (e.g., orally) a therapeutically effective amount of Compound A to the subject, for example, whereby the effective amount of Compound A reduces the dose of the opioid analgesic needed to achieve pain relief in the subject. In certain of such embodiments, the opioid analgesic is selected from buprenorphine, codeine, hydrocodone, hydromorphone, methadone, morphine, oxycodone, oxymorphone, tilidine, and tramadol, including mixtures of any of the foregoing and pharmaceutically acceptable salts of any of the foregoing. In some embodiments, the opioid analgesic is oxycodone or a pharmaceutically acceptable salt thereof, such as oxycodone HCl.

In one embodiment, the methods and uses described herein, such as the method of or use in treating pain in a subject (preferably, a mammal, such as a human) in need thereof, is achieved by administering (e.g., orally) a therapeutically effective amount of Compound A, such as from about 0.05 mg/kg to about 20 mg/kg, including from about 0.05 mg/kg to about 10 mg/kg, from about 0.1 mg/kg to about 20 mg/kg, from about 0.1 mg/kg to about 10 mg/kg, from about 0.05 mg/kg to about 5 mg/kg, from about 0.1 mg/kg to about 5 mg/kg, from about 0.05 mg/kg to about 2 mg/kg, or from about 0.1 mg/kg to about 2 mg/kg. More specific representative amounts include 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 5 mg/kg, 8 mg/kg, 10 mg/kg, 12 mg/kg, 15 mg/kg, 18 mg/kg, or 20 mg/kg or any range of amounts created by using two of the aforementioned amounts as endpoints. In some aspects, the method or use includes administering (e.g., orally) 0.1-1 mg/kg of Compound A. In certain aspects, the method includes administering (e.g., orally) 0.2-0.5 mg/kg of Compound A. In some aspects, the method or use includes administering (e.g., orally) 0.05-20 mg/kg of Compound A. In certain aspects, the method includes administering (e.g., orally) 1-10 mg/kg of Compound A.

In certain instances, the present disclosure provides a method of treating pain in a subject (preferably, a mammal, such as a human) in need thereof comprising administering (e.g., orally) a therapeutically effective amount of Compound A to the subject, wherein the pain is nociceptive pain, such as those described herein, including inflammatory pain, and wherein Compound A is administered at a dose of 0.05-5 mg/kg to the subject, such as 0.1-5 mg/kg, 0.05-2 mg/kg, or 0.1-2 mg/kg, including about 0.05 mg/kg, 0.1 mg/kg, 0.15 mg/kg, 0.2 mg/kg, 0.24 mg/kg, 0.25 mg/kg, 0.3 mg/kg, 0.35 mg/kg, 0.4 mg/kg, 0.5 mg/kg, 0.6 mg/kg, 0.7 mg/kg, 0.75 mg/kg, 0.8 mg/kg, 0.81 mg/kg, 0.85 mg/kg, 0.9 mg/kg, 1 mg/kg, 1.2 mg/kg, 1.5 mg/kg, 1.8 mg/kg, 2 mg/kg, 3 mg/kg, 4 mg/kg, or 5 mg/kg or any range of amounts created by using two of the aforementioned amounts as endpoints.

In some instances, the present disclosure provides a method of treating pain in a subject (e.g., human) in need thereof comprising administering (e.g., orally) a therapeutically effective amount of Compound A to the subject, wherein the pain is neuropathic pain, such as those described herein, and wherein Compound A is administered at a dose of 0.5-10 mg/kg to the subject, such as 0.5-8 mg/kg, 1-10 mg/kg, or 1-8 mg/kg, including about 0.5 mg/kg, 0.8 mg/kg, 1 mg/kg, 1.5 mg/kg, 2 mg/kg, 2.5 mg/kg, 2.6 mg/kg, 2.8 mg/kg, 3 mg/kg, 3.5 mg/kg, 4 mg/kg, 4.5 mg/kg, 5 mg/kg, 5.2 mg/kg, 5.5 mg/kg, 6 mg/kg, 6.5 mg/kg, 7 mg/kg, 8 mg/kg, 9 mg/kg, or 10 mg/kg or any range of amounts created by using two of the aforementioned amounts as endpoints.

In some embodiments, the methods and uses described herein, such as the method of or use in treating pain in a subject (e.g., human) in need thereof, is achieved by administering (e.g., orally) a therapeutically effective amount of Compound A, such as 2 to 200 mg of Compound A in a single or multiple dosage units. For example, the method can include administering (e.g., orally), in a single or multiple dosage units, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, or about 200 mg or administering (e.g., orally) any range of amounts created by using two of the aforementioned amounts as endpoints. In some aspects, the method or use includes oral administration of 5 to 50 mg of Compound A in a single or multiple dosage units to a subject (e.g., a human). In some aspects, the method or use includes the oral administration of 10, 20, or 25 mg of Compound A in a single or multiple dosage units to a subject (e.g., a human). In some aspects, the method or use includes oral administration of 20 mg of Compound A in a single or multiple dosage units to a subject (e.g., a human).

In some aspects, the methods and uses described herein, such as the method of or use in treating pain in a subject (e.g., human) in need thereof, is achieved by administering (e.g., orally) at least 20 mg of Compound A, such as at least 25, 30, 35, 50, 75, or 100 mg of Compound A. In some embodiments, the methods and uses described herein, such as the method of or use in treating pain in a subject in need thereof, is achieved by administering (e.g., orally) at least 50 mg of Compound A per day, such as at least 60, 75, 85, 100, 125, 150, 175, or 200 mg of Compound A per day to a subject (e.g., a human).

In some embodiments, the methods and uses described herein, such as the method of or use in treating pain in a subject (e.g., a human) in need thereof, is achieved by administering (e.g., orally) a therapeutically effective amount of Compound A per day, such as 5 to 1000 mg of Compound A per day, such as 5 to 500 mg or 5 to 250 mg of Compound A per day. For example, the method or use can include administering (e.g., orally) about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, or about 1000 mg of Compound A per day, or administering (e.g., orally) per day a range of amounts created by using two of the aforementioned amounts as endpoints. In some aspects, the method or use includes orally administering 10 to 200 mg of Compound A per day, such as 10, 15, 20, 25, 30, 35, or 40 mg to 75, 100, 125, 150, 175, or 200 mg of Compound A per day, including 20 to 150 mg per day to a subject (e.g., a human). In some aspects, the oral administration includes 50, 75, 100, or 125 mg of Compound A per day, such as 100 mg per day to a subject (e.g., a human).

In certain instances, the above daily doses of Compound A are administered (e.g., orally) as multiple doses per day, such as in two, three, four, or five doses per day. For Example, a daily dose of 100 mg, maybe administered in five 20 mg, four 25 mg, three 33.3 mg, or two 50 mg doses throughout the day.

In some embodiments, the above daily doses of Compound A are administered (e.g., orally) as a single dose. For example, about 5, 10, 15, 20, 25, or 30 mg to about 50, 65, 75, 100, 125, or 150 mg of Compound A per day can be orally administered as a single dose, including 10-25 mg, 10-30 mg, and 10-40 mg per day as a single dose, such as 10-25 mg per day as a single dose. Relatedly, any of the doses of Compound A discussed in the preceding paragraphs may be included in a single unit dosage form or in multiple unit dosage forms, such as two, three, or four unit dosage forms.

In certain embodiments, the methods and uses described herein, when using the daily dosing disclosed herein, achieve a steady state for Compound A within 6 to 9 days, such as in about 1 week.

In some embodiments, the methods and uses described herein for treating pain by administering (e.g., orally) Compound A include administering according to a 12-hour (i.e., twice-a-day), 24-hour (i.e., once-a-day), 48-hour (i.e., once-per-two-days), 72-hour, 96-hour, 5-day, 6-day, 1-week, or 2-week dosing regimen, particularly 12-hour, 24-hour, or 48-hour dosing regimens. Such regimens can involve administering any of the above-described doses or daily doses. For instance, the present disclosure provides methods of treating pain in a subject (e.g., a human) in need thereof, comprising administering (e.g., orally) a therapeutically effective amount of Compound A to the subject according to 12-hour, 24-hour, 48-hour, 72-hour, 96-hour, 5-day, 6-day, 1-week, or 2-week intervals, particularly 12-hour, 24-hour, or 48-hour intervals, wherein the amount of Compound A corresponds to any of the above-described doses or daily doses. In certain such embodiments, Compound A is orally administered to a human subject under fed conditions, e.g., from between about 30 minutes before to about 2 hour after eating a meal, including during a meal or within 15 minutes after eating a meal.

In additional embodiments, the above-discussed methods or uses of treating pain by administering a therapeutically effective amount of Compound A comprises oral administration of Compound A to a human subject under fed conditions, e.g., from between about 30 minutes before to about 2 hour after eating a meal, including during a meal or within 15 minutes after eating a meal. In some embodiments, the oral administration of Compound A to a human subject under fed conditions significantly enhances the bioavailability and exposure of Compound A as compared to the oral administration of Compound A to the subject under fasted conditions. In some embodiments, the oral administration of Compound A to a human subject under fed conditions increases one or more pharmacokinetic parameters for Compound A (e.g., Cₘₐₓ, AUC_{inf}, Tₘₐₓ, t½_{λz}, etc.) as compared to when the same amount of Compound A is orally administered to the subject under fasted conditions.

In certain embodiments, the methods and uses described herein administer Compound A in the form of a pharmaceutically acceptable oral composition that comprises Compound A and one or more pharmaceutically acceptable carriers or excipients. The amount of Compound A included in these compositions may correspond to one or more of the amounts described herein. In some embodiments, the compositions are a unit dose.

Examples of pharmaceutically acceptable oral compositions that comprise Compound A include solid formulations (such as tablets, capsules, lozenges, dragees, granules, powders, wafers, multi-particulates, and films), liquid formulations (such as aqueous solutions, elixirs, tinctures, slurries, suspensions, and dispersions), and aerosolized formulations (such as mists and sprays). In one embodiment, a pharmaceutically acceptable oral composition of Compound A includes a pediatric suspension or granulate. All above-noted amounts of Compound A may be included in such formulations, e.g., a capsule comprising 5, 10, 15, 10, 25, 30, or 35 mg of Compound A.

Examples of compositions suitable for parenteral administration of Compound A, include sterile injectable solutions, suspensions, or dispersions, including aqueous or oleaginous preparations, particularly aqueous. In some embodiments, Compound A is administered according to a method or use described herein in an injectable sterile aqueous formulation that includes a parenterally-acceptable diluent or solvent, such as water, Ringer's solution, isotonic sodium chloride solution, buffered aqueous solutions, and aqueous solutions containing a miscible alcohol, such as 1,3-butanediol. Additional suitable excipients for parenteral formulations of Compound A include, mono- or di-glycerides; fatty acids, such as oleic acid and its glyceride derivatives; natural pharmaceutically-acceptable oils, such as olive oil or castor oil, including their polyoxyethylated versions; long-chain alcohol diluents or dispersants, such as alkyl celluloses, including carboxymethyl cellulose; and surfactants, such as Tweens, Spans and other emulsifying agents or bioavailability enhancers.

In another embodiment, kits are provided for oral administration of Compound A for the treatment of pain. Such kits comprise a plurality of oral unit dosage forms of Compound A in combination with instructions for orally administering Compound A.

Additional embodiments and examples of the present disclosure are described herein. These embodiments and examples are illustrative and should not be construed as limiting the scope of the claimed invention.

### 5. EXAMPLES

Studies were conducted to determine the effect of Compound A in rodent models of pain (e.g., acetic acid induced mouse model of visceral pain and rat spinal nerve ligation/Chung model of neuropathic pain). Additional studies are conducted to determine the effect, if any, of Compound A in accepted models of pain.

### 5.1. Example 1. Acetic Acid Induced Mouse Model of Visceral Pain

Objective: The acetic acid test was performed to assess the potential efficacy of Compound A in the acetic acid writhing (AAW) model of inflammatory pain. The AAW test was performed as described previously (Yeping Bi et al., “Visceral hyperalgesia induced by forebrain-specific suppression of native Kv7/KCNQ/M-current in mice.” Mol. Pain 2011, 7:84; Gabriela F. Pavao-de-Souza et al., “Acetic acid- and phenyl-p-benzoquinone-induced overt pain-like behavior depends on spinal activation of MAP kinases, PI3K and microglia in mice.” Pharmacol. Biochem. Behav. 101 (2012) 320-328; Kazufumi Hirano et al., “Kv7.2-7.5 voltage-gated potassium channel (KCNQ2-5) opener, retigabine, reduces capsaicin-induced visceral pain in mice.” Neurosci. Lett. 413 (2007) 159-162; Mosad A. Ghareeb et al., “HPLC-ESI-MS/MS Profiling of Polyphenolics of a Leaf Extract from Alpinia zerumbet (Zingiberaceae) and Its Anti-Inflammatory, Anti-Nociceptive, and Antipyretic Activities In Vivo.” Molecules 23 (2018) 3238; and Yaroslav A. Andreev et al., “Analgesic Activity of Acid-Sensing Ion Channel 3 (ASIC3) Inhibitors: Sea Anemones Peptides Ugr9-1 and APETx2 versus Low Molecular Weight Compounds.” Mar. Drugs 16 (2018), 500).

Study Design: Briefly, acetic acid was injected intraperitoneally at 0.4% concentration in 6-7 week old CD1 mice. After the injection of acetic acid, animals were placed in a chamber and their subsequent writhing behavior was video recorded. A writhe is defined as contraction of abdominal muscles accompanied by elongation of the body and extension of hind limbs or rotation of the trunk. Writhes were counted between 5-15 min after the injection of acetic acid.

Selection of Acetic Acid Dose: Based on the concentration response (0.2-0.8%) of acetic acid in CD1 mice, EC75 concentration (0.4%) was selected to produce optimal writhing response. Diclofenac was used as positive control in the acetic acid model.

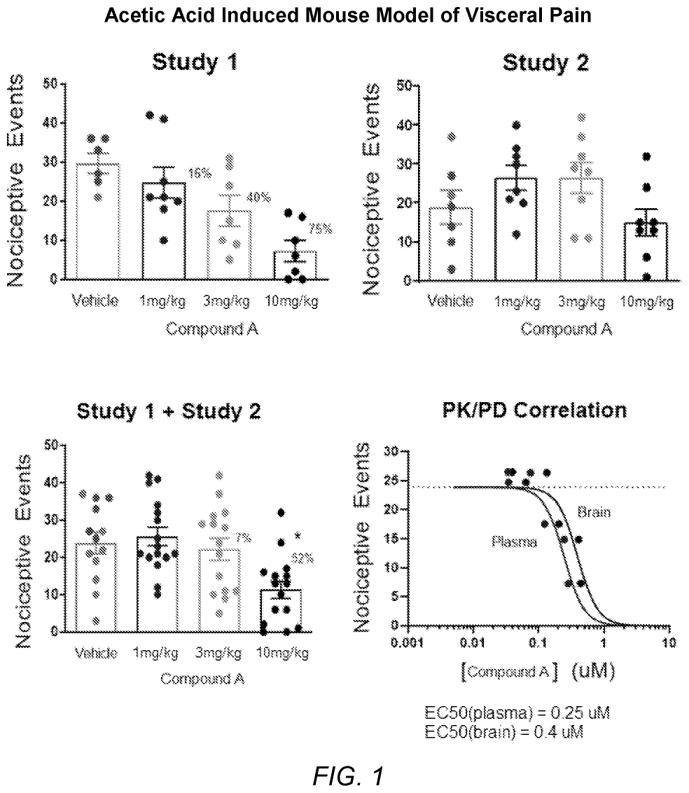

Results: The first study (FIG. 1, Study 1) shows a dose-responsive decrease in the number of nociceptive events was observed through the dose groups (vehicle, 1 mg/kg, 3 mg/kg, and 10 mg/kg Compound A). The second study (FIG. 1, Study 2) shows a decrease in the number of nociceptive events at 10 mg/kg Compound A. A PK/PD correlation was shown between Compound A concentrations in brain and plasma to the observed efficacy (FIG. 1, PK/PD Correlation). The EC₅₀ (effective concentration to give a 50% reduction in nociceptive events) was 0.25 μM and 0.4 μM in plasma and brain respectively.

### 5.2. Example 2. Spinal Nerve Ligation-Induced Model of Neuropathic Pain in Rats

Objective: The efficacy of Compound A was assessed using the rat spinal nerve ligation (SNL)/Chung model of neuropathic pain. The SNL model in rats was developed as described previously (Chung J M, Kim H K, Chung K. “Segmental spinal nerve ligation model of neuropathic pain.” Methods Mol. Med. 99 (2004) 35-45).

TABLE 1
Materials for SNL Model

| | | | Route of | | Dose | Vol. of |
|---|---|---|---|---|---|---|
| Substance | Appearance | Formulation | Admin | Ptt^{a} | expression | Admin |
| (Vehicle) | — | 0.5% (w/v) | p.o. | Once daily | mg/kg of | 5 mL/kg |
| | | CMC-Na salt | | from Day 14 | supplied | body |
| | | and 0.1% (v/v) | | to Day 18 | substance | weight |
| | | Tween 80 in | | (120 min | | |
| | | distilled water | | before the first | | |
| | | | | test on Days | | |
| | | | | 14 and 18) | | |
| Compound A | white | Dispersed in | p.o. | Once daily | mg/kg of | 5 mL/kg |
| | powder | 0.5% (w/v) | | from Day 14 | supplied | body |
| | | carboxymethyl- | | to Day 18 | substance | weight |
| | | cellulose | | (120 min | | |
| | | (CMC)-Na salt | | before the first | | |
| | | and 0.1% (v/v) | | test on Days | | |
| | | Tween 80 in | | 14 and 18) | | |
| | | distilled water | | | | |
| Retigabine | white | Dispersed in | p.o. | Once daily | mg/kg of | 5 mL/kg |
| (Selleckchem) | powder | 0.5% (w/w) | | from Day 14 | supplied | body |
| | | methylcellulose | | to Day 18 | substance | weight |
| | | and 0.2% (v/v) | | (120 min | | |
| | | Tween 80 in | | before the first | | |
| | | distilled water | | test on Days | | |
| | | | | 14 and 18) | | |
| Morphine | white | Dissolved in | p.o. | 60 min before | mg/kg of | 5 mL/kg |
| hydrochloride | powder | distilled water | | the first test on | salt | body |
| (Caesar Loretz | | | | Days 14 and | | weight |
| GmbH) | | | | 18 (distilled | | |
| | | | | water | | |
| | | | | administered | | |
| | | | | once daily | | |
| | | | | from Day 15 | | |
| | | | | to Day 17) | | |

^{a}Pretreatment time.

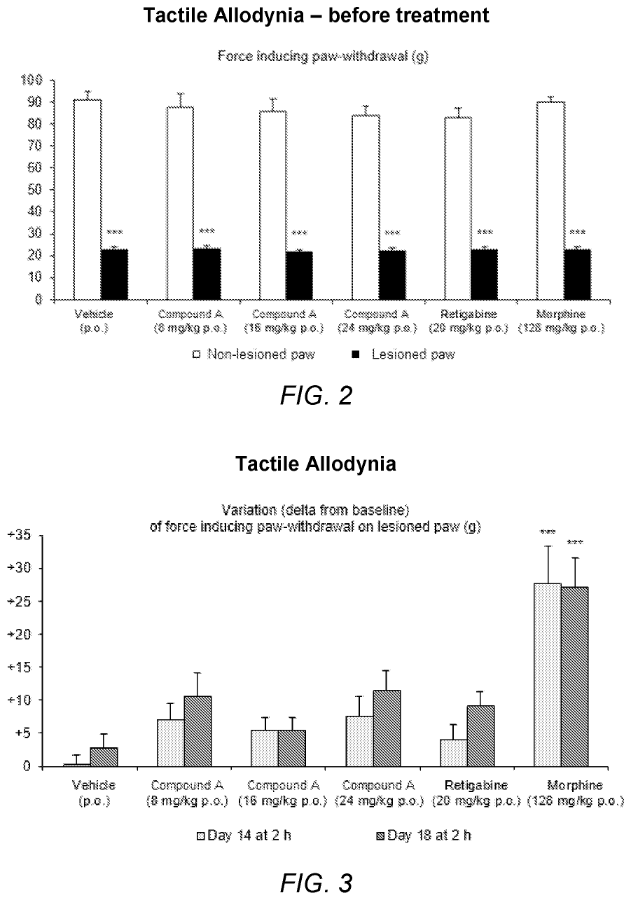

Study Design. Briefly, on Day 0, von Frey and cold plate tests were used for baseline measurements of tactile and cold allodynia respectively. Post-baseline measurements, rats (171-209 g male Sprague Dawley) were anesthetized and an incision at the L4-S2 level was performed to expose the left L5 nerve. A ligature was tied tightly around the L5 nerve (Chung et al.; R. Dost et al., “The anti-hyperalgesic activity of Retigabine is mediated by KCNQ potassium channel activation.” Naunyn-Schmiedeberg's Arch. Pharmacol. 369 (2004) 382-390; Gordon Blackburn-Munro et al., “The anticonvulsant retigabine attenuates nociceptive behaviors in rat models of persistent and neuropathic pain.” Eur. J Pharmacol. 460 (2003) 109-116; and Wu Y J et al., “Discovery of (S,E)-3-(2-fluorophenyl)-N-(1-(3-(pyridin-3-yloxy)phenyl)ethyl)-acrylamide as a potent and efficacious KCNQ2 (Kv7.2) opener for the treatment of neuropathic pain.” Bioorg Med. Chem. Lett. 23 (2013) 6188-91). The wound was then sutured and the rats were allowed to recover. On Day 13, post-surgery baselines for tactile and cold allodynia were measured to verify neuropathic pain. Animals were assigned to treatment groups based on the post-surgery baseline score. Table 2 and FIG. 2 show the results of the tactile allodynia von Frey test in rats on Day 13, prior to treatment with vehicle, Compound A, retigabine or morphine.

TABLE 2
Pretreatment Test on Day 13: Electronic von Frey Test

| | Force inducing paw-withdrawal (g) | | |
|---|---|---|---|
| | Non-lesioned paw | Lesioned paw | |
| To be treated with | Mean ± s.e.m. | Mean ± s.e.m. | Mean ± s.e.m. |
| Vehicle (p.o.) | 91.2 ± 3.7 | 22.7 ± 1.5 | −75% *** |
| Compound A (8 mg/kg p.o.) | 87.7 ± 6.4 | 23.0 ± 1.0 | −74% *** |
| Compound A (16 mg/kg p.o.) | 85.8 ± 5.7 | 21.8 ± 1.0 | −75% *** |
| Compound A (24 mg/kg p.o.) | 83.8 ± 4.4 | 22.1 ± 1.7 | −74% *** |
| Retigabine (20 mg/kg p.o.) | 82.8 ± 4.3 | 22.7 ± 1.7 | −73% *** |
| Morphine (128 mg/kg p.o.) | 90.1 ± 2.4 | 22.6 ± 1.6 | −75% *** |

Paired Student's t test: NS = Not Significant;
* = p < 0.05;
** = p < 0.01;
*** = p < 0.001

Dosing: For efficacy testing, Compound A was dosed orally once a day for 5 consecutive days from Day 14-18. Animals were evaluated again for tactile and thermal allodynia on Day 14 and Day 18 two hours after the administration of Compound A. Morphine was used as positive control. Table 3 shows the body weight of the rats over the duration of testing.

TABLE 3
Body Weight
Treatment once

| daily from Day | Body weight (g) (mean ± s.e.m.) | | | | | | |
|---|---|---|---|---|---|---|---|
| 14 to Day 18^{a} | Day 0 | Day 13 | Day 14 | Day 15 | Day 16 | Day 17 | Day 18 |
| Vehicle (p.o.) | 194.4 ± 2.0 | 309.4 ± 4.1 | 314.5 ± 3.7 | 327.1 ± 3.8 | 335.2 ± 4.2 | 345.6 ± 4.5 | 351.6 ± 4.7 |
| Compound A | 194.6 ± 2.4 | 307.4 ± 6.6 | 311.5 ± 6.7 | 327.8 ± 7.8 | 338.2 ± 7.6 | 347.0 ± 8.2 | 354.0 ± 8.0 |
| (8 mg/kg | | | | | | | |
| p.o.) | | | | | | | |
| Compound A | 190.6 ± 2.5 | 296.8 ± 4.0 | 300.2 ± 5.1 | 314.9 ± 5.3 | 320.2 ± 5.2 | 330.1 ± 6.7 | 334.9 ± 6.4 |
| (16 mg/kg | | | | | | | |
| p.o.) | | | | | | | |
| Compound A | 192.2 ± 1.8 | 296.8 ± 4.5 | 301.4 ± 5.1 | 319.8 ± 5.7 | 328.3 ± 5.5 | 334.2 ± 6.4 | 339.1 ± 5.5 |
| (24 mg/kg | | | | | | | |
| p.o.) | | | | | | | |
| Retigabine | 190.6 ± 3.3 | 297.8 ± 5.0 | 300.8 ± 4.8 | 312.6 ± 5.1 | 321.1 ± 5.2 | 330.6 ± 5.3 | 336.8 ± 5.7 |
| (20 mg/kg | | | | | | | |
| p.o.) | | | | | | | |
| Morphine | 188.4 ± 2.0 | 293.9 ± 4.7 | 295.9 ± 4.6 | 312.8 ± 5.5 | 302.0 ± 5.4 | 298.9 ± 5.4 | 306.7 ± 5.2 |
| (128 mg/kg | | | | | | | |
| p.o.) | | | | | | | |

^{a}Treatment once on Day 14 and on Day 18 for group treated with morphine.

### 5.2.1 Tactile Allodynia Evaluation Using the Electronic Von Frey Test

Rats were placed under an inverted acrylic plastic box on a grid floor. The tip of an electronic von Frey probe was then applied with increasing force to the surgery hind paw and the force required to induce paw-withdrawal was automatically recorded. The procedure was carried out 3 times and the mean paw withdrawal force was calculated. The experimenter was double blinded to treatment.

Results: Tables 4-5 and FIG. 3 show the results of the tactile allodynia von Frey evaluation on Day 14 and Day 18. Compared to morphine, neither Compound A nor retigabine had much effect on the tactile allodynia endpoint. This was not surprising for the Kv7.2 mechanism (see Blackburn-Munro and Jensen, Eur J Pharmacol, 460(2-3): 109-116 (2003)).

TABLE 4
Tactile Allodynia Evaluation on Day 14: Electronic von Frey Test on Lesioned Paw

| | Force inducing paw-withdrawal (g) | | | | | |
|---|---|---|---|---|---|---|
| Treatment once | | | Day 14 at 2 h: Delta from baseline | | | |
| daily from Day | Baseline on Day 14 | Day 14 at 2 h | | Compared with Vehicle (p.o.) | | |
| 14 to Day 18^{a} | Mean ± s.e.m. | Mean ± s.e.m. | Mean ± s.e.m. | c | d | Δ |
| Vehicle (p.o.) | 22.9 ± 1.2 | 23.2 ± 1.7 | +0.3 ± 1.4 | | | |
| Compound A | 21.5 ± 1.6 | 28.5 ± 3.4 | +7.0 ± 2.5 | NS | — | +6.7 |
| (8 mg/kg | | | | | | |
| p.o.) | | | | | | |
| Compound A | 19.9 ± 1.9 | 25.2 ± 1.9 | +5.4 ± 2.1 | | — | +5.1 |
| (16 mg/kg | | | | | | |
| p.o.) | | | | | | |
| Compound A | 22.4 ± 1.4 | 29.9 ± 2.9 | +7.5 ± 3.1 | | — | +7.2 |
| (24 mg/kg | | | | | | |
| p.o.) | | | | | | |
| Retigabine | 22.5 ± 1.6 | 26.5 ± 2.5 | +4.0 ± 2.2 | | — | +4.0 |
| (20 mg/kg | | | | | | |
| p.o.) | | | | | | |
| Morphine | 23.0 ± 1.8 | 50.6 ± 5.0 | +27.7 ± 5.6 | *** | | +27.4 |
| (128 mg/kg | | | | | | |
| p.o.)^{b} | | | | | | |

Inter-group comparison:
NS = Not Significant;
* = p < 0.05;
** = p < 0.01;
*** = p < 0.001.
^{a}Treatment once on Day 14 and on Day 18 for group treated with morphine.
^{b}Test 1 h after administration.
c For test substances-treated groups: One-way ANOVA with group as factor.
d Dunnett's test when One-way ANOVA is significant.

TABLE 5
Tactile Allodynia Evaluation on Day 18: Electronic von Frey Test on Lesioned Paw

| | Force inducing paw-withdrawal (g) | | | | | |
|---|---|---|---|---|---|---|
| Treatment once | | | Day 18 at 2 h: Delta from baseline | | | |
| daily from Day | Baseline on Day 14 | Day 18 at 2 h | | Compared with Vehicle (p.o.) | | |
| 14 to Day 18^{a} | Mean ± s.e.m. | Mean ± s.e.m. | Mean ± s.e.m. | c | d | Δ |
| Vehicle (p.o.) | 22.9 ± 1.2 | 25.7 ± 1.8 | +2.8 ± 2.1 | | | |
| Compound A | 21.5 ± 1.6 | 32.1 ± 3.6 | +10.6 ± 3.5 | NS | — | +7.8 |
| (8 mg/kg | | | | | | |
| p.o.) | | | | | | |
| Compound A | 19.9 ± 1.9 | 25.2 ± 2.0 | +5.4 ± 2.1 | | — | +2.6 |
| (16 mg/kg | | | | | | |
| p.o.) | | | | | | |
| Compound A | 22.4 ± 1.4 | 33.8 ± 3.6 | +11.5 ± 3.0 | | — | +8.7 |
| (24 mg/kg | | | | | | |
| p.o.) | | | | | | |
| Retigabine | 22.5 ± 1.6 | 31.8 ± 2.2 | +9.2 ± 2.1 | | — | +6.4 |
| (20 mg/kg | | | | | | |
| p.o.) | | | | | | |
| Morphine | 23.0 ± 1.8 | 50.0 ± 4.5 | +27.1 ± 4.6 | *** | | +24.3 |
| (128 mg/kg | | | | | | |
| p.o.)^{b} | | | | | | |

Inter-group comparison:
NS = Not Significant;
* = p < 0.05;
** = p < 0.01;
*** = p < 0.001.
^{a}Treatment once on Day 14 and on Day 18 for group treated with morphine.
^{b}Test 1 h after administration.
c For test substances-treated groups: One-way ANOVA with group as factor.
d Dunnett's test when One-way ANOVA is significant.

### 5.2.2 Thermal Allodynia Evaluation Using the Cold Plate Test

The cold plate apparatus was maintained at 4±1° C. and was surrounded by an acrylic glass enclosure. Rats were placed on the plate for a period of 5 minutes. The latency to 1^{st} paw-withdrawal, total number of paw withdrawals, and total duration of withdrawal responses were recorded for the surgery hind paw.

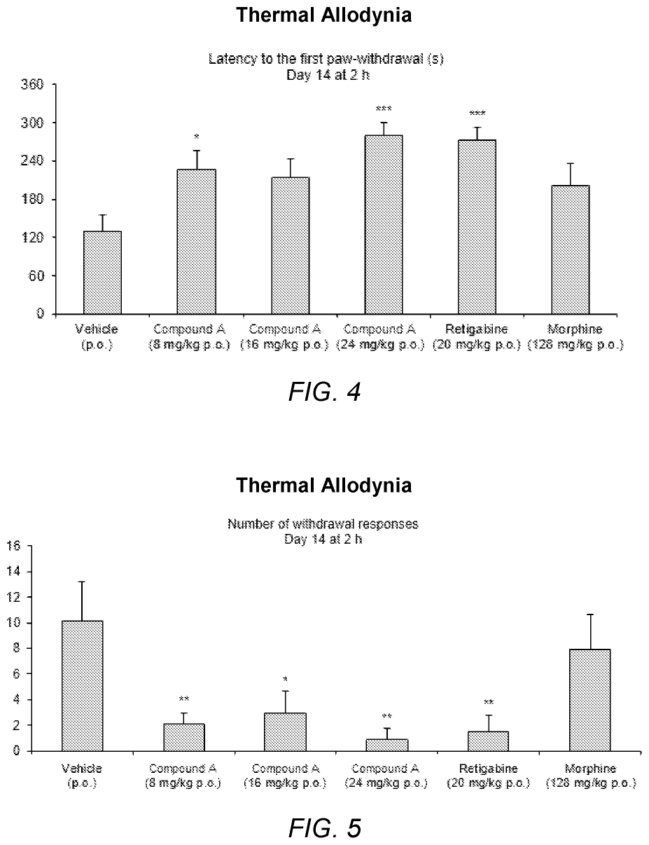

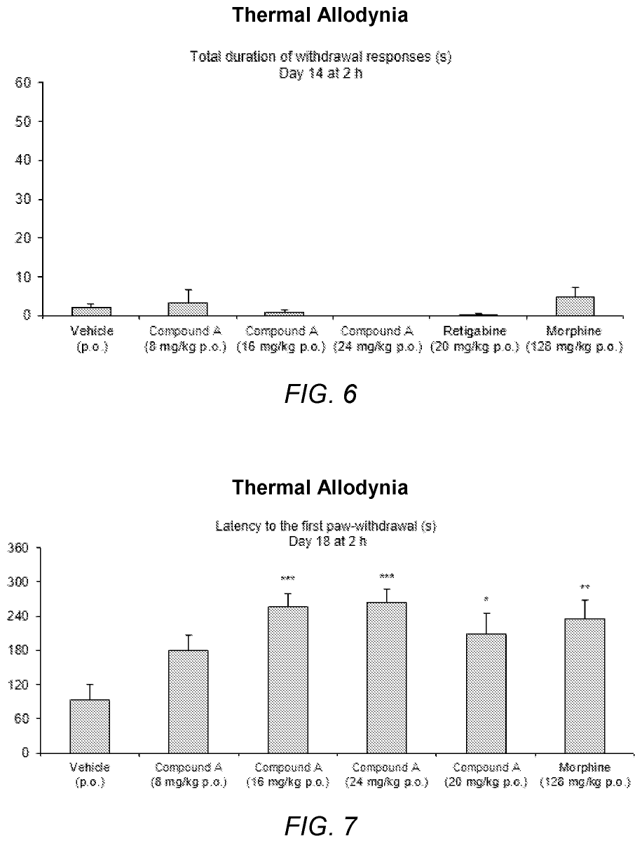

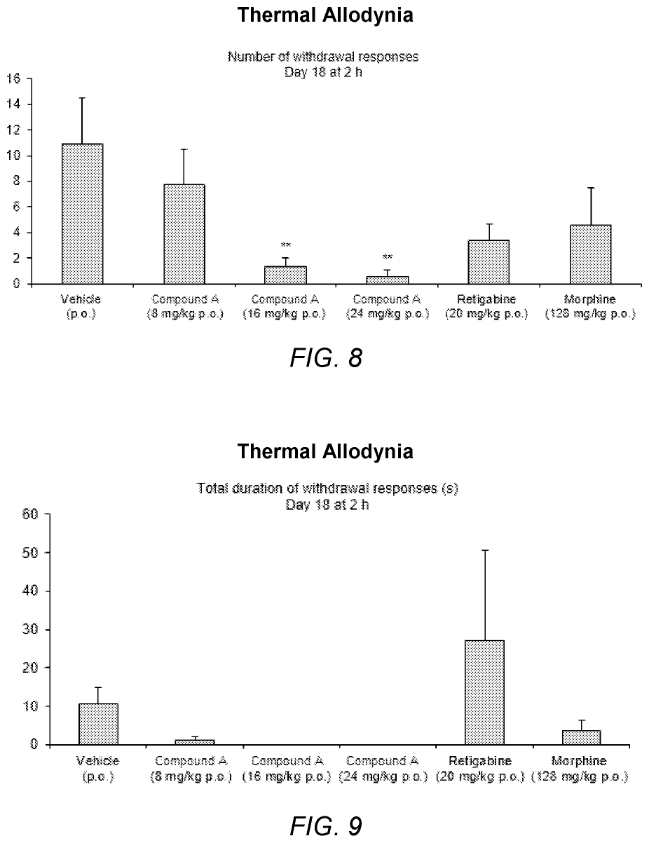

Results: Tables 6-7 and FIGS. 4-9 show the results of the thermal allodynia von Frey evaluation on Day 14 and Day 18. FIGS. 7-9 show that at both 16 and 24 mg/kg, Compound A outperformed morphine (128 mg/kg) in the thermal allodynia endpoint.

TABLE 6
Thermal Allodynia Evaluation on Day 14: Cold Plate Test on Lesioned Paw

| | Latency to first | | | Number of withdrawal | | | | | Total duration of | | | |
|---|---|---|---|---|---|---|---|---|---|---|---|---|
| | paw-withdrawal (s) | | | responses | | | | | withdrawal responses (s) | | | |
| | Day 14 at 2 h | | | | | | | | | | | |
| | | | | | | Compared | | | | | | |
| Treatment once | | Compared with | | | | with Vehicle | | | | Compared with | | |
| dailyfrom Day | Mean ± | Vehicle (p.o.) | | | Mean ± | (p.o.) | | | Mean ± | Vehicle (p.o.) | | |
| 14 to Day 18^{a} | s.e.m. | C | d | Δ% | s.e.m. | c | d | Δ % | s.e.m. | c | d | Δ% |
| Vehicle (p.o.) | 130.0 ± 25.5 | | | | 10.1 ± 3.1 | | | | 2.2 ± 1.0 | | | |
| Compound A | 226.8 ± 29.4 | *** | * | +74 | 2.1 ± 0.8 | ** | ** | −79 | 3.4 ± 3.4 | NS | — | +55 |
| (8 mg/kg | | | | | | | | | | | | |
| p.o.) | | | | | | | | | | | | |
| Compound A | 214.9 ± 29.2 | | NS | +65 | 3.0 ± 1.7 | | * | −70 | 0.8 ± 0.7 | | — | −64 |
| (16 mg/kg | | | | | | | | | | | | |
| p.o.) | | | | | | | | | | | | |
| Compound A | 280.0 ± 20.0 | | *** | +115 | 0.9 ± 0.9 | | ** | −91 | 0.0 ± 0.0 | | — | −100 |
| (24 mg/kg | | | | | | | | | | | | |
| p.o.) | | | | | | | | | | | | |
| Retigabine | 272.7 ± 20.1 | | *** | +110 | 1.5 ± 1.3 | | ** | −85 | 0.3 ± 0.3 | | — | −86 |
| (20 mg/kg | | | | | | | | | | | | |
| p.o.) | | | | | | | | | | | | |
| Morphine | 201.3 ± 35.8 | NS | | +55 | 7.9 ± 2.8 | NS | | −22 | 4.9 ± 2.4 | NS | | +123 |
| (128 mg/kg | | | | | | | | | | | | |
| p.o.)^{b} | | | | | | | | | | | | |

Inter-group comparison:
NS = Not Significant;
* = p < 0.05;
** = p < 0.01;
*** = p < 0.001.
^{a}Treatment once on Day 14 and on Day 18 for group treated with morphine.
^{b}Test 1 h after administration.
c For test substances-treated groups: One-way ANOVA with group as factor. For comparison-treated group: Unpaired Student's test.
d Dunnett's test when One-way ANOVA is significant.

TABLE 7
Thermal Allodynia Evaluation on Day 18: Cold Plate Test on Lesioned Paw

| | Latency to first | | | Number of withdrawal | | | | | Total duration of | | | |
|---|---|---|---|---|---|---|---|---|---|---|---|---|
| | paw-withdrawal (s) | | | responses | | | | | withdrawal responses (s) | | | |
| Treatment | Day 18 at 2 h | | | | | | | | | | | |
| once daily | | Compared with | | | | Compared with | | | | Compared with | | |
| from Day 14 | Mean ± | Vehicle (p.o.) | | | Mean ± | Vehicle (p.o.) | | | Mean ± | Vehicle (p.o.) | | |
| to Day 18^{a} | s.e.m. | C | d | Δ% | s.e.m. | c | d | Δ% | s.e.m. | c | d | Δ% |
| Vehicle (p.o.) | 94.2 ± 25.6 | | | | 10.9 ± 3.5 | | | | 10.8 ± 4.3 | | | |
| Compound A | 180.4 ± 26.5 | *** | NS | +92 | 7.7 ± 2.8 | ** | NS | −29 | 1.3 ± 0.9 | NS | — | −88 |
| (8 mg/kg | | | | | | | | | | | | |
| p.o.) | | | | | | | | | | | | |
| Compound A | 257.0 ± 23.5 | | *** | +173 | 1.3 ± 2.8 | | ** | −88 | 0.0 ± 0.0 | | — | −100 |
| (16 mg/kg | | | | | | | | | | | | |
| p.o.) | | | | | | | | | | | | |
| Compound A | 263.8 ± 24.3 | | *** | +180 | 0.6 ± 0.5 | | ** | −94 | 0.0 ± 0.0 | | — | −100 |
| (24 mg/kg | | | | | | | | | | | | |
| p.o.) | | | | | | | | | | | | |
| Retigabine | 209.3 ± 36.1 | | * | +122 | 3.4 ± 1.3 | | NS | −69 | 27.3 ± 23.5 | | — | +153 |
| (20 mg/kg | | | | | | | | | | | | |
| p.o.) | | | | | | | | | | | | |
| Morphine | 235.8 ± 33.2 | ** | | +150 | 4.6 ± 2.8 | NS | | −58 | 3.8 ± 2.7 | NS | | −65 |
| (128 mg/kg | | | | | | | | | | | | |
| p.o.)^{b} | | | | | | | | | | | | |

Inter-group comparison:
NS = Not Significant;
* = p < 0.05;
** = p < 0.01;
*** = p < 0.001.
^{a}Treatment once on Day 14 and on Day 18 for group treated with morphine.
^{b}Test 1 h after administration.
c For test substances-treated groups: One-way ANOVA with group as factor. For comparison-treated group: Unpaired Student's test.
d Dunnett's test when One-way ANOVA is significant.

### 5.3. Example 3. Crossover Study with Pharmacokinetic Analysis

The pharmacokinetics (PK), safety, and tolerability of single doses of Compound A in healthy right-handed male human subjects were investigated in a randomized, double-blind, placebo-controlled, transcranial magnetic stimulation (TMS) crossover study.

An objective of the study was to evaluate the safety, tolerability, and pharmacokinetics of single doses of Compound A in healthy male subjects.

Twenty healthy right-handed male subjects were enrolled and randomized in a blinded fashion to receive a single oral dose of 20 mg Compound A or placebo (1:1 randomization ratio) on Day 1, then were crossed over to receive a single dose of the other treatment on Day 7.

Subjects were screened within 27 days prior to entering the study on Day 1. For Period 1, subjects were admitted to the study unit and dosed on Day 1, and discharged on Day 2. For Period 2, following a washout of 6 days, the same subjects were again admitted to the study unit and dosed on Day 7, and discharged on Day 8. All subjects returned to the clinical unit for an outpatient visit on Day 14, and received a follow-up telephone call on Day 37.

Subjects were dosed in a fed state, but the timing of dosing relative to meals was changed during the study, and varied between a high fat or standard meal eaten either 2 h or 30 minutes prior to dosing, and a high fat or standard meal eaten 1 h or 2.5 h after dosing.

PK variables included maximal plasma concentration (Cₘₐₓ), time of maximal plasma concentration (Tₘₐₓ), terminal elimination half-life (t_{1/2}), elimination rate constant (λz), area under the curve from 0 to 24 h (AUC₀₋₂₄ₕ), area under the curve from time zero to the last quantifiable concentration (AUC₀₋ₜₗₐₛₜ), area under the curve from time zero to infinity (AUC_{0-inf}), the percentage of AUC that is due to extrapolation from tlast to infinity (% AUCₑₓₜᵣₐₚ), apparent total body clearance following oral administration (CL/F), CL/F normalized by body weight, mean residence time from time zero to the last quantifiable concentration (MRTₗₐₛₜ), mean residence time extrapolated to infinity (MRT_{inf}), apparent volume of distribution during the terminal phase (Vz/F), and Vz/F normalized by body weight.

### 5.3.1. Pharmacokinetic Analysis

The PK parameters for this study were summarized in two ways. Firstly, PK parameters were calculated where possible using the PK samples collected during each 24 h sampling period for Period 1 and Period 2 separately. Secondly, PK parameters were determined using samples beyond the 24 h sampling period (i.e., from Day 7/8 and/or Day 14). For subjects who received Compound A in the first period, PK samples taken prior to placebo treatment provided additional PK timepoints at >24 h. For subjects who received Compound A in the second period, there was no >24 h PK timepoint until a Day 14 PK sample was added. Thus, subjects randomized to receive Compound A in the second period who were enrolled prior to implementation of the additional PK sample at Day 14 did not have PK data beyond 24 h. The full PK profile data set consists of the 16 subjects for whom PK samples were taken at >24 h post-dose. For discussion of the PK parameters below, the full PK profile data set was generally used, because it allowed more accurate estimation of PK parameters.

Initially, subjects were dosed 2 hours after a high fat meal with a relatively high fat lunch provided 1 hour after dosing. After blinded review of the PK profiles in the initial 8 subjects, the fat content of the lunch was reduced in an attempt to reduce the time to Tₘₐₓ. In addition, the timing of the meal relative to dose was changed from 2 hours prior, to 30 minutes prior to dose and subsequently the fat content of the breakfast was reduced. The timing and type of meal for each subject is specified in Table 8. Overall, there was no clear difference in Cₘₐₓ or Tₘₐₓ despite the changes in meal composition and timing relative to dose. As such the PK data is presented without categorization according to meal content, or relative timing of the meal.

TABLE 8
Type and Timing of Meals Relative to Dosing

| | Pre-dose Meal | | Post-dose Meal | |
|---|---|---|---|---|
| Subjects | Type | Time | Type | Time |
| 901, 908, | High fat^{a} | 2 h pre-dose | High fat | 1 h post-dose |
| 910, 907 | | | | |
| 912, 919, | High fat | 2 h pre-dose | Standard | 1 h post-dose |
| 914, 918 | | | | |
| 927, 925, | High fat | 0.5 h pre-dose | Standard | 2.5 h post-dose |
| 928, 924 | | | | |
| 930, 934, | Standard | 0.5 h pre-dose | Standard | 2.5 h post-dose |
| 933, 937, | | | | |
| 938, 941, | | | | |
| 940, 942 | | | | |

^{a}Except Subject 910 had standard breakfast prior to dosing

### 5.3.1.1. Plasma Concentrations

A plasma concentrations over time for the full PK profile were recorded. At the 2 h, 4 h and 6 h timepoints, the mean SD plasma concentrations were 15.9±21.4 ng/mL, 30.2±21.1 ng/mL and 42.1±19.1 ng/mL, respectively.

There was no difference in mean Cₘₐₓ or Tₘₐₓ between periods (Table 9). The overall time to peak plasma concentrations ranged from 1.9 to 12 h, with a median time of 7.8 h.

Subjects who received placebo in Period 2 had low but measurable Compound A levels at the start of the placebo treatment period, with a mean Cₘₐₓ of 5.84 ng/mL (range 3.34-9.61 ng/mL).

TABLE 9
Pharmacokinetic Parameters by Period, Overall, and for Full PK Profile

| | | 20 mg Compound A | | | |
|---|---|---|---|---|---|
| | | | | | Full PK |
| | | Period 1 | Period 2 | Overall | Profile |
| Parameter | Statistic | (N = 10) | (N = 10) | (N = 20) | (N = 16) |
| Cₘₐₓ (ng/mL) | Mean ± SD | 60.2 ± 17.3 | 58.3 ± 9.94 | 59.2 ± 13.8 | 60.1 ± 14.9 |
| | Range | 29.9-77.1 | 46.2-79.4 | 29.9-79.4 | 29.9-79.4 |
| Tₘₐₓ (h) | Median | 6.94 | 7.83 | 7.83 | 6.83 |
| | Range | 1.92-12 | 1.92-8.15 | 1.92-12 | 1.92-12 |
| AUC₀₋₂₄ (ng*h/mL) | Mean ± SD | 693 ± 184 | 681 ± 142 | 687 ± 160 | 692 ± 151 |
| | Range | 383-951 | 358-869 | 358-951 | 383-951 |
| Cₗₐₛₜ (ng/mL) | Mean ± SD | 16.4 ± 5.61 | 16.4 ± 3.87 | 16.4 ± 4.69 | 4.52 ± 1.82 |
| | Range | 10.1-27.8 | 7.1-21.3 | 7.1-27.8 | 1.33-7.67 |
| Tₗₐₛₜ (h) | Mean ± SD | 23.8 ± 0.375 | 23.8 ± 0.213 | 23.8 ± 0.299 | 235 ± 81.5 |
| | Range | 23.1-24.3 | 23.5-24.1 | 23.1-24.3 | 142-360 |
| T_{1/2} (h) | Mean ± SD | 11.4 ± 2.6 | 10.6 ± 2.9 | 11.1 ± 2.6 | 127 ± 84.6 |
| | Range | 8.46-14.9 | 8.01-14.3 | 8.01-14.9 | 48.2-306 |

### 5.3.1.2. Other Pharmacokinetic Parameters for Full PK Profile

A summary of other PK parameters is provided in Table 10. The mean AUCₗₐₛₜ was 2370 ng*h/mL, which included PK samples from follow-up visits when available. The AUC_{inf} from the same data set was 3155 ng*h/mL and the median (range) extrapolated area was 19.9% (range 10.6-40.5%). This relatively high level of extrapolated area in some subjects suggests that the parameters calculated from λz (such as half-life, MRT_{inf}, clearance, and volume of distribution) should be analyzed with caution and may have higher inherent variance in their calculation.

The mean normalized volume of distribution (Vz/F) of 16.3 L/kg was well above total blood volume for the mean body weight of 72.3 kg, indicating that the drug distributes out of plasma into surrounding tissues.

Body weight normalized clearance (CL/F) was 97.5 mL/h/kg (equivalent to approximately 1.6 mL/min/kg). This value is plasma clearance, not blood clearance; however, even adjusting for hematocrit, it is well below total hepatic blood flow of 17 mL/min/kg (Carlisle et al., Gut 1992, 33:92-97), suggesting a low extraction drug.

TABLE 10
Pharmacokinetic Parameters (Full PK Data Set)

| | 20 mg Compound A Full PK Profile | |
|---|---|---|
| | Data Set (N = 16) | |
| Parameter | Mean ± SD | Range |
| AUCₗₐₛₜ (ng*h/mL) | 2370 ± 680 | 1583-4400 |
| AUC_{inf} (ng*h/mL) | 3155 ± 1341 | 1923-7393 |
| Vz/F normalized (L/kg) | 16.3 ± 9.06 | 6.4-33.8 |
| t_{1/2} (h) | 127 ± 84.6 | 48.2-306 |
| MRTₗₐₛₜ (h) | 77.4 ± 23.7 | 48.2-122 |
| MRT_{inf} (h) | 102 ± 84.8 | 33-304 |
| CL/F normalized (mL/h/kg) | 97.5 ± 25.7 | 40.3-136 |

### 5.3.2. Pharmacokinetic Conclusions

Compound A was slowly absorbed after a 20 mg oral dose with median peak plasma concentrations occurring approximately 8 hours after administration. Upon absorption, it distributed out of plasma into surrounding tissues and was slowly cleared from systemic circulation at rates well below hepatic blood flow, indicating minimal hepatic extraction (metabolism). It exhibited a mean half-life of 127 h (range 48.2-306 h) and mean residence time of 102 h (range 33-304 h) which may be an underestimation since a number of subjects had % AUCₑₓₜᵣₐₚ values above 20% and as high as 40%.

Washout between periods was not long enough to allow Compound A levels fall below the limit of quantitation in subjects who received placebo in Period 2 (mean 3.1 ng/mL, range 1.3-6.8 ng/mL).

All of the U.S. patents, U.S. patent application publications, U.S. patent applications, foreign patents, foreign patent applications, and non-patent publications referred to in this specification, including U.S. Provisional Application No. 62/945,093, filed Dec. 6, 2019 and 62/948,010, filed Dec. 13, 2019, are incorporated herein by reference in their entireties.

Although the foregoing compositions, methods, and uses have been described in some detail to facilitate understanding, it will be apparent that certain changes and modifications may be practiced within the scope of the appended claims. Accordingly, the described embodiments are to be considered as illustrative and not restrictive, and the claimed invention is not to be limited to the details given herein, but may be modified within the scope and equivalents of the appended claims.

1. A method of treating pain in a human in need thereof, comprising administering a therapeutically effective amount of Compound A to the human;
wherein Compound A is N-[4-(6-fluoro-3,4-dihydro-1H-isoquinolin-2-yl)-2,6-dimethylphenyl]-3,3-dimethylbutanamide.

2. The method of claim 1, wherein Compound A is administered to the human in combination with an opioid analgesic.

3. A method of reducing the dose of an opioid analgesic administered to a human in need thereof, comprising administering a therapeutically effective amount of Compound A to the human, whereby the effective amount of Compound A reduces the dose of the opioid analgesic needed to achieve pain relief in the human;
wherein Compound A is N-[4-(6-fluoro-3,4-dihydro-1H-isoquinolin-2-yl)-2,6-dimethylphenyl]-3,3-dimethylbutanamide.

4. The method of claim 2, wherein the opioid analgesic is selected from buprenorphine, codeine, hydrocodone, hydromorphone, methadone, morphine, oxycodone, oxymorphone, tilidine, and tramadol, including mixtures of any of the foregoing and pharmaceutically acceptable salts of any of the foregoing.

5. The method of claim 1, wherein the method comprises enhancing the opening of a Kv7 potassium channel in the human.

6. A method of enhancing the opening of a Kv7 potassium channel in a human, comprising administering an effective amount of Compound A to the human;
wherein Compound A is N-[4-(6-fluoro-3,4-dihydro-1H-isoquinolin-2-yl)-2,6-dimethylphenyl]-3,3-dimethylbutanamide; and
wherein the human is suffering from pain.

7. (canceled)

8. The method of claim 6, wherein the method is selective for enhancing the opening of a Kv7 potassium channel selected from one or more of Kv7.2, Kv7.3, Kv7.4, or Kv7.5 over Kv7.1.

9. (canceled)

10. The method of claim 1, wherein the pain is nociceptive pain, neuropathic pain, or a combination thereof.

11. The method of claim 10, wherein the pain is nociceptive pain.

12-15. (canceled)

16. The method of claim 10, wherein the pain is neuropathic pain.

17. (canceled)

18. The method of claim 1, wherein the pain is acute pain.

19. The method of claim 1, wherein the pain is chronic pain.

20. (canceled)

21. The method of claim 1, wherein the pain is moderate or severe pain.

22. (canceled)

23. The method of claim 1, wherein the pain is of tumor origin.

24. (canceled)

25. The method of claim 1, wherein the pain is breakthrough pain

26. The method of claim 1, wherein the pain is associated with a migraine, including migraine without aura (“common migraine”), migraine with aura (“classic migraine”), migraine without headache, basilar migraine, familial hemiplegic migraine, migrainous infarction, migraine with prolonged aura, or a combination thereof.

27. The method of claim 1, wherein Compound A is orally administered to the human.

28. The method of claim 1, wherein Compound A is administered at a dose of 2 to 200 mg to the human.

29-35. (canceled)

36. The method of claim 1, wherein Compound A is administered at a dose of 5-1000 mg per day to the human.

37-46. (canceled)

47. The method of claim 1, wherein Compound A is orally administered to the human from between about 30 minutes before to about 2 hours after eating a meal.

48. (canceled)
